(19) United States
(12) Patent Application Publication
(10) Pub. No.: US 2019/0252038 A1
(43) Pub. Date: Aug. 15, 2019

(54) METHODS FOR QUANTIFICATION OF POLYNUCLEOTIDES

(71) Applicant: UNIVERSITY OF PITTSBURGH-OF THECOMMONWEALTH SYSTEM OF HIGHER EDUCTION, Pittsburgh, PA (US)

(72) Inventors: Anthony CILLO, Pittsburgh, PA (US); John MELLORS, Pittsburgh, PA (US)

(21) Appl. No.: 16/335,778

(22) PCT Filed: Sep. 22, 2017
(86) PCT No.: PCT/US2017/052955

§ 371 Date: Mar. 22, 2019

Related U.S. Application Data
(60) Provisional application No. 62/398,730, filed on Sep. 23, 2016.

(51) Int. Cl. G16B 20/10; C12Q 1/6851; C12Q 1/70; G01N 21/64

(52) U.S. Cl. CPC G16B 20/10; G01N 21/6428; C12Q 1/703; C12Q 1/6851

(57) ABSTRACT

Systems, methods, and media for quantifying the amount of polynucleotide in a sample. In some embodiments, methods can include obtaining a plurality of PCR assay quantification results of viral polynucleotide found in a given sample and then converting the results at or below the assay's lower detection limit into estimated polynucleotide copies per millimeter of sample based at least on the fraction of replicate outcomes that are positive results, the fraction of replicates that have a quantified value, and a nominal continuous viremia estimate.

### STATEMENT OF GOVERNMENT FUNDING

This invention was made with Government support under Grant No. AI113102 awarded by the National Institutes of Health. The Government has certain rights in the invention.

### BACKGROUND OF THE INVENTION

The field of the invention is infectious disease diagnostics. Levels of HIV-1 RNA in plasma (viremia) can readily be measured by commercial assays during uncontrolled HIV-1 infection, however the limit of quantification of commercially available assays often precludes the use of viremia as an endpoint in clinical trials in those on suppressive antiretroviral therapy (ART). Specifically, the limit of quantification of Roche COBAS AmpliPrep/TAQMAN v.2.0 (“CAP/CTM”) is 20 copies per milliliter of plasma, yet patients on suppressive ART have viremia in the range of less than 1 to 3 copies per milliliter of plasma on average.

While there is a specialized research laboratory assay (i.e., the single copy assay, known as SCA) that can be used to quantify viremia below the clinical limit of quantification, SCA is too complex to be scaled up for large clinical trials. Further, only a handful of virology laboratories globally can perform SCA.

Accordingly, there is a need in the art for methods of quantifying that are able to provide an estimate, by prediction or inference, of HIV-1 RNA, and polynucleotides generally, in a sample, that is more accurate than current commercial assays (e.g., CAP/CTM) and substantially comparable in accuracy to SCA.

### SUMMARY OF THE DISCLOSURE

Systems, methods, and media for quantifying the amount of polynucleotide in a sample are provided. In some examples or embodiments, the polynucleotide is RNA. In still further examples or embodiments, the RNA is HIV-1 RNA. An example method includes a) obtaining, by the computing system, a plurality of quantification results of a polynucleotide in the sample from an assay having a lower limit of detection; b) generating, by the computing system, a first parameter value by determining the fraction of the results that have a positive result; c) generating, by the computing system, a second parameter value by determining the fraction of results that have a positive and quantified result; d) generating, by the computing system, a third parameter value by generating a nominal continuous viremia estimate for the sample and calculating the logarithm base 10 of the nominal continuous viremia estimate; e) generating, by the computing system, a predicted number of polynucleotide copies per milliliter of the sample based on a combination of at least: the first parameter value, the second parameter value, and the third parameter value: and f) storing the predicted number in a database.

In some embodiments, the method comprises obtaining, by a computing system, a plurality of quantitative PCR quantification results of HIV-RNA found in the sample; generating, by the computing system, a first parameter value by determining the fraction of the quantification results that have an outcome that is positive, generating, by the computing system, a second parameter value by determining the fraction of quantification results that have an outcome that is positive and quantified; generating, by the computing system, a third parameter value by generating a nominal continuous viremia estimate for the sample and calculating the logarithm base 10 of the nominal continuous viremia estimate, generating, by the computing system, a predicted viremia estimate value for the sample based on a combination of at least: the first parameter value, the second parameter value, and the third parameter value; and storing the predicted viremia estimate value in a database.

In some embodiments, the method comprises obtaining, by a computing system, a plurality of Roche COBAS AmpliPrep/TAQMAN v.2.0 (referred to herein as “CAP/CTM”) quantification results of HIV-RNA found in the sample; generating, by the computing system, a first parameter value by determining the fraction of the CAP/CTM quantification results that have an outcome that is positive; generating, by the computing system, a second parameter value by determining the fraction of CAP/CTM quantification results that have an outcome that is positive and quantified; generating, by the computing system, a third parameter value by generating a nominal continuous viremia estimate for the sample and calculating the logarithm base 10 of the nominal continuous viremia estimate; generating, by the computing system, a predicted viremia estimate value for the sample based on a combination of at least: the first parameter value, the second parameter value, and the third parameter value: and storing the predicted viremia estimate value in a database.

### BRIEF DESCRIPTION OF THE DRAWINGS

The accompanying drawings, where like reference numerals refer to identical or functionally similar elements throughout the separate views, together with the detailed description below, are incorporated in and form part of the specification, and serve to further illustrate embodiments of concepts that include the claimed disclosure, and explain various principles and advantages of those embodiments.

The methods and systems disclosed herein have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

FIG. 1 shows an example derivation of modeling parameters for the predictive algorithm in using the R software language.

FIG. 2 is a graph showing an example fit of the modeled algorithm to fraction <20 or ≥20 by CAP/CTM and mega-iSCA.

FIG. 3 is a graph showing an example fit of the modeled algorithm to nominal continuous viremia estimate values and mega-iSCA.

FIG. 4 is a flowchart showing an example method for quantifying the amount of HIV-1 RNA in a sample via a computing system in accordance with the present disclosure.

FIG. 5 shows a block diagram of an example computing system that can implement the exemplary methods disclosed herein.

### DETAILED DESCRIPTION OF THE INVENTION

Provided herein are improved methods of quantifying, by prediction or inference, the amount of a polynucleotide (the number of copies of a polynucleotide) in a sample. In some embodiments, the sample is a liquid. Terms used throughout this application are to be construed with ordinary and typical meaning to those of ordinary skill in the art. However, Applicant desires that the following terms be given the particular definition as defined below.

As used in the specification and claims, the singular form “a,” “an,” and “the” include plural references unless the context clearly dictates otherwise. For example, the term “a cell” includes a plurality of cells, including mixtures thereof.

The terms “about” and “approximately” are defined as being “close to” as understood by one of ordinary skill in the art. In one non-limiting embodiment, the terms are defined to be within 10%. In another non-limiting embodiment, the terms are defined to be within 5%. In still another non-limiting embodiment, the terms are defined to be within 1%.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

The term “disease” refers to an abnormal condition of a part, organ, or system of a subject resulting from various causes, such as infection, inflammation, environmental factors, or genetic defect, and characterized by an identifiable group of signs, symptoms, or both. In some embodiments, the disease is an infectious disease.

“Mammal” for purposes of diagnosis refers to any animal classified as a mammal, including human, domestic and farm animals, nonhuman primates, and zoo, sports, or pet animals, such as dogs, horses, cats, cows, etc.

The terms “polynucleotide” and “oligonucleotide” are used interchangeably, and refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. A polynucleotide is composed of a specific sequence of four nucleotide bases: adenine (A); cytosine (C): guanine (G); thymine (T); and uracil (U) for thymine (T) when the polynucleotide is RNA. Thus, the term “polynucleotide sequence” is the alphabetical representation of a polynucleotide molecule.

The term “quantification result” refers herein to the result of an attempt to detect and quantify one or more items in an assay sample. It should be understood that “quantification result” refers to a positive result and/or a negative result. A “positive result” can be quantified or unquantified. Both a negative result and a positive but unquantified result can arise due to the amount of polynucleotide in the sample being less than the lower limit of quantifiable detection of the polynucleotide detection assay that is employed. For example, the lower limit of quantifiable detection of the CAP/CTM assay is twenty (20) polynucleotides per milliliter. A positive but unquantified CAP/CTM result could therefore indicate any number of polynucleotides less than twenty polynucleotides per milliliter in a sample, whereas a negative result could indicate no polynucleotides per milliliter or less than 20 polynucleotides per milliliter in the sample. Accordingly, a “negative result” is an assay output variable in which the assay detects no discernable target polynucleotide in a sample. A “positive but unquantified result” is an assay output variable in which the assay detects at least some target polynucleotide in a sample, but the amount of target polynucleotide cannot be quantified because the amount of target polynucleotide present in the sample is below the quantifiable detection limit of the assay. A “positive and quantified result” in an assay output variable in which the assay detects at least some target polynucleotide and can further quantify the amount of target polynucleotide because the amount of target polynucleotide present in the sample is at or above the quantifiable detection limit of the assay. Collectively, assay results which are below the quantifiable limit of detection of the assay are “unquantified results.” Thus, unquantified results include both negative results and positive but unquantified results.

The term “sample” refers herein to a quantitative assay sample. In some embodiments, the assay comprises a polymerase chain reaction (PCR). In other or further embodiments, the assay comprises detection or attempted detection of one or more fluorescent molecules, which number of one or more fluorescent molecules corresponds directly with the number of polynucleotide copies.

The term “subject” is defined herein to include animals such as mammals, including, but not limited to, primates (e.g., humans), cows, sheep, goats, horses, dogs, cats, rabbits, rats, mice and the like. In some embodiments, the subject has or had a viral or bacterial infection. In some embodiments, the infective virus is a Human Immunodeficiency Virus. In some embodiments, the subject has been treated with suppressive antiretroviral therapy (ART). In some embodiments, the subject is a human.

One objective of the present disclosure is to provide a predictive algorithm that can convert a plurality of polynucleotide quantification results from an assay (e.g., a quantitative polymerase chain reaction (PCR) assay), which include results under the quantitative limit of detection of the assay (“unquantified results”), into a predicted value of polynucleotide copies in a volume of the sample (“predicted value”). Unquantified results include both negative results and positive but unquantified results. It is a surprising finding of the present disclosure that the method provided herein, also referred to herein as the “predictive algorithm,” is capable of quantifying low amounts of polynucleotides in samples that were previously unquantifiable by prior art methods. In some embodiments, the methods described herein can predictably quantify less than 20 polynucleotide molecules per milliliter. In other or further embodiments, the methods described herein can predictably quantify less than 18, 15, 13, 10, 8, 5, 3 or 1 polynucleotide molecules per milliliter. Accordingly, in some embodiments, unquantified results indicate less than 20 polynucleotide copies per milliliter. In other embodiments, unquantified results indicate less than 40 polynucleotide copies per milliliter.

In some embodiments, the assay is a quantitative PCR (qPCR) assay. PCR involves making a cDNA that corresponds to a polynucleotide in the sample volume. The cDNA comprises a detectable label, such as, for example, a fluorescent label. In these examples or embodiments, the amount of the polynucleotide in the sample volume, or the number of copies of the polynucleotide in the sample volume, is determined through detection and quantification of the label. In these embodiments, the lower limit of quantifiable detection may be attributable to and/or associated with the identity of the label(s), the PCR primer(s), the PCR probe(s), and/or PCR reaction conditions.

The sample described herein can be of any type. In some embodiments, the sample is a blood plasma sample. It should be understood that the term “sample” includes dilutions thereof. For example, a plasma sample includes dilutions of the plasma sample. The polynucleotide can be either RNA or DNA. In some embodiments or examples, the polynucleotide is a viral RNA. Viral RNA includes, but is not limited to, HIV (Human Immunodeficiency Virus) RNA and HCV (Hepatitis C Virus) RNA. In some embodiments or examples, the plurality of quantification results is ten or greater. In other embodiments or examples, the plurality of quantifications results is five or greater. In still other embodiments or examples, the plurality of quantification results is greater than five, six, seven, eight, nine, or ten.

In some embodiments or examples, the PCR assay includes use and/or detection of one or more fluorescent molecules including, but not limited to, 6-carboxyfluorescein (FAM), tetrachlorofluorescein (TET). HEX, Cy 3, Cy 3.5, Cy 5, Cy 5.5, Cy 7, tetramethylrhodamine, ROX and JOE. In some embodiments or examples, the PCR assay further includes the use of one or more fluorescence quencher molecules including, but not limited to, Dabcyl, BHQ-1, BHQ-2, BHQ-3, and BBQ-650. In some embodiments or examples, the PCR assay employs one or more COBAS TAQMAN probes and/or primers including, but not limited to, primers targeting both gag and long terminal repeat regions of the HIV genome.

The PCR assay can be, in some embodiments, any quantitative PCR (qPCR) assay which amplifies and quantitates DNA in a sample. In some embodiments, the qPCR assay can comprise use of Roche COBAS TAQMAN (“CTM”), or specifically COBAS TAQMAN v.2.0. In some or further embodiments, the qPCR assay can comprise use of COBAS AmpliPrep (“CAP”). In some or further embodiments, the qPCR assay can comprise use of Hologic Aptima HIV-1 Quant Dx assay. In some or further embodiments, the qPCR assay can comprise use of Abbot RealTime HIV−1 assay. In some embodiments, the qPCR assay is selected from CAP/CTM. Hologic Aptima HIV-1 Quant Dx assay, and Abbot RealTime HIV-1 assay.

One example of a PCR assay employing one or more COBAS TAQMAN probes and primers is a CAP/CTM assay. As noted above, the CAP/CTM assay has a lower limit of quantifiable detection of 20 polynucleotide copies per milliliter. As used herein, a negative result of the CAP/CTM assay can also be referred to as “target not detected” (TND). As used herein, a positive but unquantified result of the CAP/CTM assay can also be referred to as “<20”. As used herein, a positive and quantified result of the CAP/CTM assay can also be referred to as “≥20” for which a continuous number value is provided as an assay output variable. Thus, unquantified results of a CAP/CTM assay can be indicated as “TND” and “<20”, whereas positive results of a CAP/CTM assay can be indicated as “<20” and “≥20”. Positive and quantified results are indicated by a number value, e.g., “34.”

In some embodiments, the qPCR assay has a lower limit of quantifiable detection of 30 polynucleotide copies per milliliter. A negative result of the qPCR assay can also be referred to as “target not detected” (TND). A positive but unquantified result of the qPCR assay can also be referred to as “<30”. As used herein, a positive and quantified result of the qPCR assay can also be referred to as “≥30” for which a continuous number value is provided as an assay output variable. Thus, unquantified results of a qPCR assay can be indicated as “TND” and “<30”, whereas positive results of a qPCR assay can be indicated as “<30” and “≥30”. Positive and quantified results are indicated by a number value, e.g., “34.” In some embodiments, the qPCR assay having a lower limit of quantifiable detection of 30 polynucleotide copies per milliliter is a Hologic Aptima HIV-1 Quant Dx assay.

In some embodiments, the qPCR assay has a lower limit of quantifiable detection of 40 polynucleotide copies per milliliter. A negative result of the qPCR assay can also be referred to as “target not detected” (TND). A positive but unquantified result of the qPCR assay can also be referred to as “<40”. As used herein, a positive and quantified result of the qPCR assay can also be referred to as “≥40” for which a continuous number value is provided as an assay output variable. Thus, unquantified results of a qPCR assay can be indicated as “TND” and “<40”, whereas positive results of a qPCR assay can be indicated as “<40” and “≥40”. Positive and quantified results are indicated by a number value, e.g., “44.” In some embodiments, the qPCR assay having a lower limit of quantifiable detection of 40 polynucleotide copies per milliliter is an Abbot RealTime HIV-1 assay.

It should be understood that although the remainder of the specification focuses on the application of the predictive algorithm to CAP/CTM quantification results, the present disclosure is not limited to CAP/CTM based tests. Other assays (e.g., quantitative PCR assays) having limits of detection as known to those having ordinarily skill in the art may be used with the present invention.

In developing the predictive algorithm, the following modeling parameters were discovered to generate predicted values from a CAP/CTM assay having multiple sample replicates that are comparable to that of a laborious mega-iSCA assay:
Fraction of replicate outcomes that are positive results of the CAP/CTM assay (e.g., outcomes designated as <20 polynucleotide copies per milliliter of plasma, and outcomes designated by a quantified value that is ≥20 polynucleotide copies per milliliter of plasma);
Fraction of replicates that are quantitative values of the CAP/CTM assay (i.e. ≥20 polynucleotide copies per milliliter of plasma); and
Nominal continuous viremia estimate.
   The nominal continuous viremia estimate takes into account the quantitative value of the ≥20 results by averaging results from all replicates. This estimate also assigns TND (negative results) a value of 5 polynucleotide copies per milliliter of plasma, <20 (positive but unquantified results) a value of 10 polynucleotide copies per milliliter of plasma, and assigns the continuous numerical values (e.g., 34) from CAP/CTM assay results for ≥20 (positive and quantified results) copies per milliliter of plasma. This parameter was used purely for modeling purposes, and was not an estimate of true low-level viremia.

In some embodiments, the value assigned to negative results (e.g., a result of “TND”) in calculating the nominal continuous viremia estimate ranges from 0 to 10. In some embodiments, the value assigned to TND is from greater than 0 to 10. In some embodiments, the value assigned to TND is from 1 to 9, from 2 to 8, from 3 to 7, or from 4 to 6. In some embodiments, the value assigned to TND is 0, 1, 2, 3, 4, 5, 6, 7, 8, 9, or 10. Preferably, the value assigned to TND is 0. In other preferred embodiments, the value assigned to TND is 5.

In some embodiments, the value assigned to positive but unquantified results (e.g., <20 polynucleotide copies per milliliter of plasma) in calculating the nominal continuous viremia estimate is from greater than 0 to less than 20. In some embodiments, the value assigned to positive but unquantified results ranges from 1 to 19, from 2 to 18, from 3 to 17, from 4 to 16, from 5 to 15, from 6 to 14, from 7 to 13, from 8 to 12, or from 9 to 11. In some embodiments, the value assigned to positive but unquantified results is 0, 1, 2, 3, 4, 5, 6, 7, 8, 9, 10, 11, 12, 13, 14, 15, 16, 17, 18, 19, or 20. Preferably, the value assigned to positive but unquantified results is 5. In other preferred embodiments, the value assigned to positive but unquantified results is 10.

The positive and quantified results of a CAP/CTM assay are quantified values ≥20, which are assigned as continuous numerical values in calculating the nominal continuous viremia estimate. While qPCR assays can detect very high levels of viral loads (e.g., up to 10,000,000 polynucleotide copies or more), the herein disclosed methods are preferably used to predictably quantify low levels of viral loads. In some embodiments, the continuous numerical value from CAP/CTM results range from 20 to 200 polynucleotide copies per milliliter of plasma. In some embodiments, the continuous numerical value from CAP/CTM results range from 20 to 175, from 20 to 150, from 20 to 125, from 20 to 100, from 20 to 75, or from 20 to 50 polynucleotide copies per milliliter of plasma. In some embodiments, the methods further comprise identifying and removing a continuous numerical value which is a statistical outlier from other continuous numerical values of the same subject.

The present invention is not limited to the input values above which may be specific to the CAP/CTM assay. The predictive algorithm described may utilize input values from a range of replicate assays from other systems (e.g., other qPCR assays including, but not limited to: Hologic Aptima HIV-1 Quant Dx and Abbot RealTime HIV-1 assays) that are representative of the following three types of results: a negative result (i.e., no detectable polynucleotides present), a positive but unquantified result (a detectable but not quantifiable result or an ordinal set of detectable but not quantifiable results), and a positive and quantified result (a detectable and quantifiable result or a set of detectable and quantifiable results).

The predictive algorithm was developed and tested for accuracy in the following manner. First, results of a CAP/CTM assay were obtained. In particular, twenty-one HIV-1 patients on long term suppressive ART underwent plasmapheresis or large volume blood draws to obtain plasma. A total of ten or eleven 1.1 milliliter replicates of plasma were assayed by CAP/CTM. The CAP/CTM assay first isolated polynucleotide from plasma within the AmpliPrep platform, followed by real-time PCR on the TAQMAN system. This system is completely automated, and the only steps required to be completed manually were pipetting portions of the sample into tubes to be loaded into the AmpliPrep, and moving the extracted tubes from the AmpliPrep to the TAQMAN. Potential CAP/CTM outcomes included target not detected (TND), <20 (positive for HIV-1 RNA but not quantified), and ≥20 (positive for HIV-1 RNA and quantified). Next, results of a single copy assay (SCA) targeting the integrase region of the HIV-1 genome (iSCA) were obtained. The iSCA assay can quantify viremia below the CAP/CTM assay limit of quantification. The iSCA assay was performed using a large-volume adaptation of SCA (termed mega-iSCA) on plasma. Mega-iSCA involved ultracentrifugation of up to 40 milliliters of plasma to pellet virions, followed by isolation of nucleic acid with in-house reagents. After nucleic acid was isolated, PCR was performed to quantify levels of HIV−1 RNA. The results of the CAP/CTM and mega-iSCA assays are provided in Table 1 below, wherein “PID” refers to the personal identification number of each subject providing samples, and “cps/mL” refers to polynucleotide copies per milliliter of sample. CAP/CTM results are shown for each replicate sample provided by each subject.

TABLE 1

| | | | | | | | | | | | | Mega- |
|---|---|---|---|---|---|---|---|---|---|---|---|---|
| | | | | | | | | | | | | iSCA |
| PID | CAP/CTM Results | | | | | | | | | | | (cps/mL) |
| 1 | TND | TND | TND | 23 | TND | TND | TND | TND | TND | TND | — | 0.09 |
| 2 | TND | <20 | TND | TND | TND | TND | <20 | TND | TND | TND | — | 0.28 |
| 3 | TND | TND | <20 | TND | TND | TND | TND | TND | <20 | TND | <20 | 0.37 |
| 4 | TND | <20 | <20 | <20 | <20 | TND | <20 | <20 | <20 | <20 | 36 | 2.2 |
| 5 | TND | TND | TND | TND | TND | TND | TND | TND | TND | TND | <20 | <0.046 |
| 6 | 30 | TND | <20 | <20 | <20 | TND | <20 | TND | <20 | TND | — | 1.5 |
| 7 | TND | TND | TND | TND | TND | TND | TND | TND | TND | TND | — | <0.05 |
| 8 | TND | TND | TND | TND | TND | TND | TND | TND | TND | TND | — | <0.046 |
| 9 | 22 | TND | <20 | TND | <20 | <20 | <20 | TND | <20 | <20 | — | 1.6 |
| 10 | TND | TND | TND | <20 | TND | TND | TND | TND | TND | TND | — | 0.092 |
| 11 | TND | <20 | TND | TND | TND | <20 | TND | <20 | <20 | <20 | — | 0.18 |
| 12 | <20 | TND | <20 | TND | TND | <20 | TND | <20 | TND | <20 | — | 1.7 |
| 13 | <20 | <20 | TND | <20 | 28 | <20 | 22 | 27 | 37 | <20 | — | 1.2 |
| 14 | 50 | TND | <20 | TND | <20 | <20 | 38 | TND | TND | 68 | — | 1.7 |
| 15 | <20 | TND | TND | TND | TND | TND | <20 | <20 | TND | <20 | — | 1.4 |
| 16 | TND | TND | TND | TND | TND | TND | TND | <20 | TND | TND | — | <0.092 |
| 17 | <20 | <20 | <20 | TND | <20 | <20 | <20 | <20 | <20 | <20 | — | 6.3 |
| 18 | <20 | <20 | TND | TND | <20 | <20 | <20 | TND | TND | <20 | — | 2.1 |
| 19 | TND | 26 | TND | TND | <20 | <20 | TND | <20 | TND | TND | — | 2.3 |
| 20 | <20 | TND | TND | TND | TND | TND | TND | TND | <20 | TND | — | <0.092 |
| 21 | <20 | TND | <20 | <20 | TND | TND | TND | TND | TND | <20 | — | 2.9 |

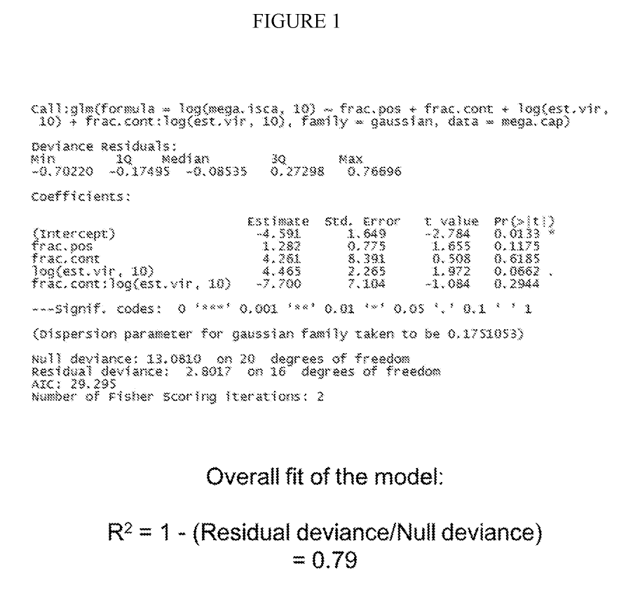

Afterwards, as shown in FIG. 1, modeling parameters for the predictive algorithm were identified, and the viability of the parameters were confirmed by generalized linear modeling using the R Software language. In FIG. 1, as understood by those having ordinary skill in the art, the “˜” symbol is used is in the R software as a generalized linear model function call that takes the form “y˜ model”, where y is a response of a linear model. The “+” operator is used in the R software to denote a term in the series of terms of the model. The “:” operator is used in the form “first:second” to indicate a set of terms generated by taking the interaction of all terms in the first with all terms in the second. In general, when a “glm” function is called for a given input data set, the R software fits the generalized linear model to the input data set, and then outputs an estimated intercept and estimated parameter coefficients that correspond to a series of parameters.

It was found that the combination of the following four parameters obtained from results of a CAP/CTM assay are able to suitably model the results of a mega-iSCA assay: a) “frac.pos”, b) “frac.cont”, c) “log(cst.vir,10)”, and d) “frac.cont:log(est.vir,10)” using the input data “mega.cap” in which:

a) “frac.pos” refers to the fraction of replicate outcomes that are positive results of the CAP/CTM assay (e.g., outcomes designated as <20 polynucleotide copies per milliliter of plasma, and outcomes designated by a quantified value that is 220 polynucleotide copies per milliliter of plasma);

b) “frac.cont” refers to the fraction of replicate outcomes that are quantitative values of the CAP/CTM assay (i.e. ≥20 polynucleotide copies per milliliter of plasma)

c) “log(est.vir, 10)” is a log-base 10 input of the dataset “est.vir.”, where “est.vir” refers to the nominal continuous viremia estimate described in detail further below; and

d) “frac.cont:log(est.vir,10)” is an interaction of the dataset “frac.cont” with a log-base 10 of the dataset “est.vir.”

The term “mega.cap” refers to results obtained from replicate plasma samples that have been run on the Roche CAP/CTM platform. The use of the term as part of the function call indicates to R software the location for a dataset that contains at least the “frac.pos”, “frac.cont”. “est.vir” data.

It was further found that the parameter values can be combined with the following estimate coefficient values and an estimated intercept value to generate a sufficiently accurate estimate of the number of polynucleotide copies found per millimeter of plasma.

The estimated intercept (herein, “I₀”) can range from about −6.24 to about −2.942. In some aspects, the estimated intercept can range from about −6.0 to about −3.5, from about −5.5 to about −4.0, or from about −5.0 to about −4.5.

The estimated coefficient for the “frac.pos” parameter (herein, “a₁”) can range from about 0.527 to about 2.037. In some embodiments, the estimated coefficient for the “frac.pos” parameter can range from about 0.7 to about 1.9, from about 0.9 to about 1.7, or from about 1.2 to about 1.4.

The estimated coefficient for the “frac.cont” parameter (herein, “a₂”) can range from about −4.13 to about 12.652. In some embodiments, the estimated coefficient for the “frac.cont” parameter can range from about −2.0 to about 10.0, from about 0.0 to about 8.0, from about 2.0 to about 6.0, or from about 3.5 to about 4.5.

The estimated coefficient for the “log(est.vir, 10)” parameter (herein, “a₃”) can range from about 2.2 to about 6.73. In some embodiments, the estimated coefficient for the “log(est.vir, 10)” parameter can range from about 2.5 to about 6.5, from about 3.0 to about 6.0, from about 3.5 to about 5.5, or from about 4.0 to about 5.0.

The estimated coefficient for the interaction between the “frac.cont” parameter and the “log(est.vir, 10),” which is denoted as “frac.cont:log(est.vir, 10)” (herein, “a₂:a₃”) in the R software, can range from about −14.804 to about −0.596. In some embodiments, the “frac.cont:log(est.vir, 10)” can range from about −13 to about −2, from about −11 to about −4, from about −9 to about −6, or from about −8 to about −7.

As shown in FIG. 1, when the “glm” function as called based on the data found in Table 1, the estimated intercept (“I₀”) was found to be −4.591, the estimated coefficient for the “frac.pos” parameter (“a₁”) was found to be 1.282, the estimated coefficient for the “frac.cont” parameter (“a₂”) was found to be 4.261, the estimated coefficient for the “log(est.vir, 10)” parameter (“a₃”) was found to be 4.65, and the estimated coefficient for the interaction between “frac.cont” and “log(est.vir, 10)” (“a₂:a₃”) was found to be −7.700.

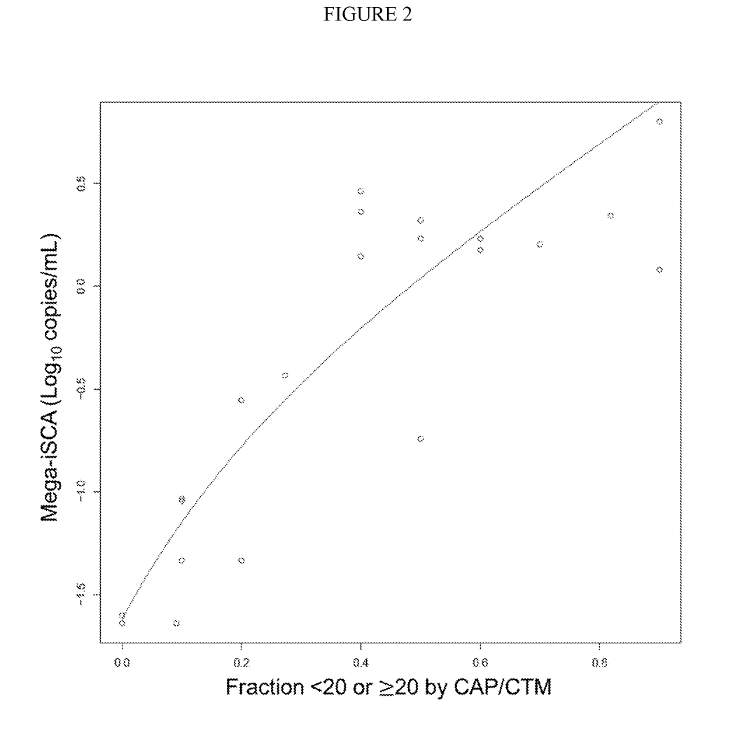

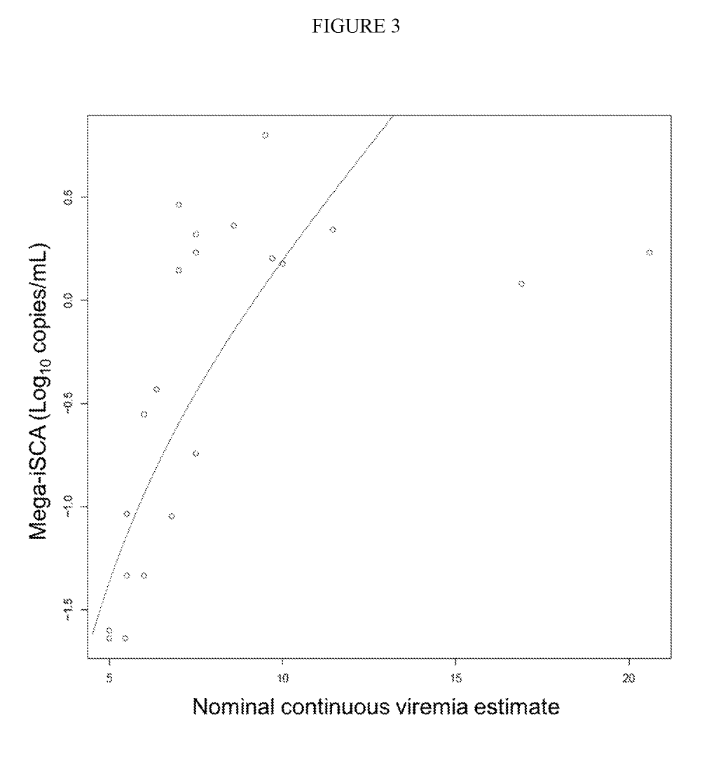

FIG. 2 shows the fit of the modeled algorithm to the fraction <20 or ≥20 as determined by CAP/CTM and mega-iSCA. FIG. 3 shows the fit of the modeled algorithm to the nominal continuous viremia estimate as determined by CAP/CTM. Both FIGS. 2 and 3 show that the modeled algorithm fit the data well.

Table 2 shows a comparison of results from the predictive algorithm and results from the mega-iSCA analysis. The median fold-change between assays was 0.82, and the interquartile range for fold-change between assays was 0.67 to 2.0. CAP/CTM was below the limit of detection in 2 of 21 participants, while mega-iSCA was below the limit of detection for 5 of 21 participants.

TABLE 2

| | | | Fold-change |
|---|---|---|---|
| | Predicted | Mega-iSCA | (Mega-iSCA versus |
| | CAP/CTM results | results | Predicted |
| PID | (cps/mL) | (cps/mL) | CAP/CTM) |
| 1 | 0.11 | 0.09 | 0.82 |
| 2 | 0.14 | 0.28 | 2.0 |
| 3 | 0.22 | 0.37 | 1.7 |
| 4 | 6.8 | 2.2 | 0.32 |
| 5 | 0.070 | <0.046 | 0.35 |
| 6 | 2.0 | 1.5 | 0.75 |
| 7 | <0.034 | <0.05 | 0.74 |
| 8 | <0.034 | <0.046 | 0.68 |
| 9 | 2.4 | 1.6 | 0.67 |
| 10 | 0.070 | 0.092 | 1.3 |
| 11 | 0.91 | 0.18 | 0.20 |
| 12 | 0.91 | 1.7 | 1.9 |
| 13 | 0.93 | 1.2 | 1.3 |
| 14 | 1.9 | 1.7 | 0.90 |
| 15 | 0.50 | 1.4 | 2.8 |
| 16 | 0.070 | <0.092 | 0.66 |
| 17 | 8.5 | 6.3 | 0.74 |
| 18 | 0.91 | 2.1 | 2.3 |
| 19 | 0.63 | 2.3 | 3.6 |
| 20 | 0.14 | <0.092 | 0.33 |
| 21 | 0.50 | 2.9 | 5.8 |

These data demonstrate that the relationship between mega-iSCA and the output values from multiple replicates of CAP/CTM is complex. These data also show that, although single measures of CAP/CTM do not provide accurate measures of low-level viremia, multiple replicates of CAP/CTM can be used to predict the amount of virus in plasma based on the techniques described herein. The present disclosure identifies for the first time that the level of viremia below the lower limit of detection of the current CAP/CTM assay can be accurately inferred from a unique proprietary algorithm derived using data from mega-iSCA and multiple replicates of the CAP/CTM assay. This innovative method to quantify low-level viremia is scalable for large clinical trials.

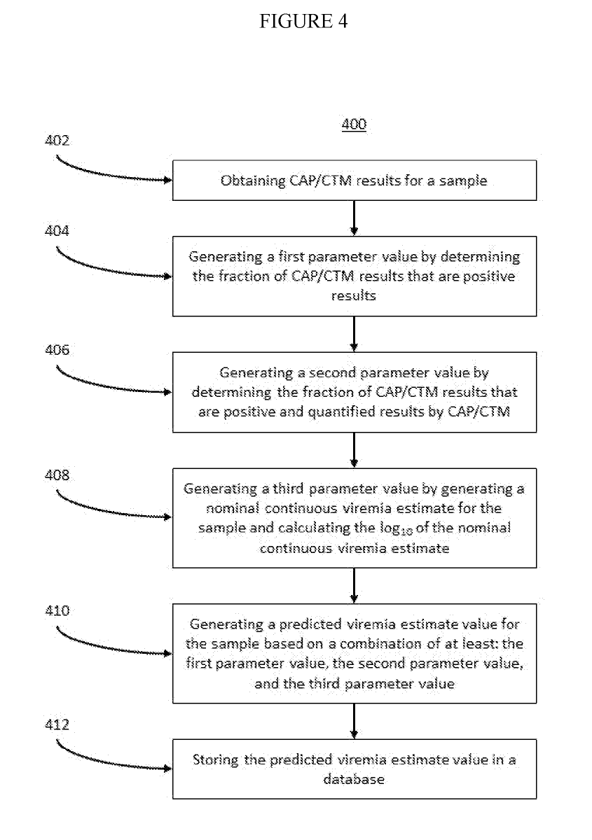

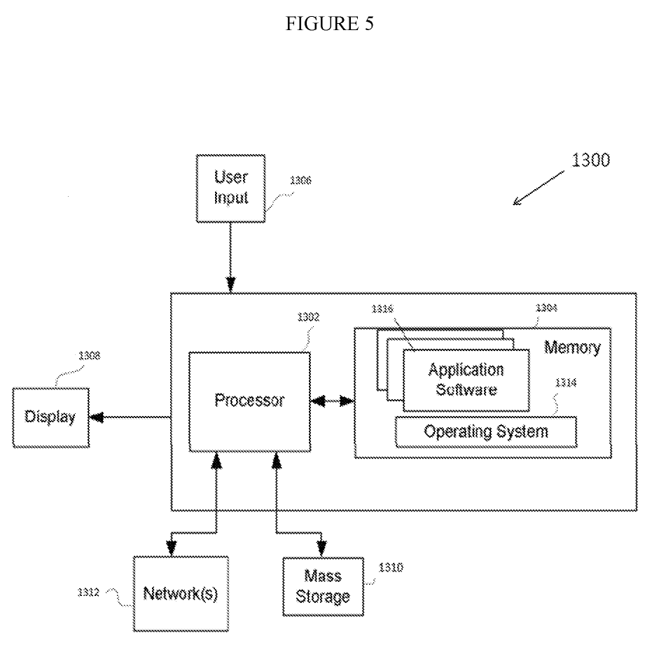

FIG. 4 provides an example method 400 for quantifying the amount of HIV-1 RNA in a sample via a computing system using the parameters described above (e.g., “frac.pos”, “frac.cont”, “log(est.vir,10)”, “frac.cont:log(est.vir,10)”). An exemplary computing system includes a processor configured to execute program instructions for performing the steps of method 400. The program instructions may be stored in any suitable computer-readable medium. An exemplary computing system is more fully described with reference to FIG. 5. The method 400 includes the computing system obtaining a plurality of CAP/CTM quantification results of HIV-1 RNA found in a given sample (step 402) and then converting the CAP/CTM results (i.e., TND, <20, and quantified values of polynucleotide copies detected) into polynucleotide copies per millimeter of plasma in the various manners described below (steps 404-412). The CAP/CTM quantification results can be obtained by the computing system via communication with a database for example. The database can be stored locally on the computing system or at a remote location.

At step 404, the computing system generates a first parameter value (i.e., “frac.pos”) by determining the fraction of the CAP/CTM results that are positive (e.g., outcomes designated as <20 polynucleotide copies per milliliter of plasma, and outcomes designated by a quantified value that is ≥20 polynucleotide copies per milliliter of plasma). At step 406, the computer system generates a second parameter value (e.g., “frac.cont”) by determining the fraction of the CAP/CTM results that are quantitative values of the CAP/CTM assay (i.e. ≥20 polynucleotide copies per milliliter of plasma).

At step 408, a third parameter value is generated by generating a nominal continuous viremia estimate for the sample, and calculating the log₁₀ of the nominal continuous viremia estimate (e.g., “log(est.vir,10)”). In certain embodiments, the nominal continuous viremia estimate is generated by a) assigning a value of 5 polynucleotide copies per milliliter of plasma to any result that has an outcome of TND (a negative result), b) assigning a value of 10 polynucleotide copies per milliliter of plasma to any result that has a positive but unquantified outcome (i.e. <20 polynucleotide copies per millimeter of plasma); c) assigning a continuous value from CAP/CTM for ≥20 polynucleotide copies per milliliter of plasma results; and d) averaging all the assigned values. Thus, when the computing system analyzes, for example, the CAP/CTM results of PID 13 of Table 1, the following values would be assigned from left to right: 10, 10, 5, 10, 28, 10, 22, 27, 37, and 10. Accordingly, the nominal continuous viremia for said row would be 16.9 polynucleotide copies per millimeter of plasma. Thus, the third parameter value for the PID 13 (e.g., “log(est.vir,10)”) would be the log-base 10 value of the nominal continuous viremia estimate, which is about 1.228 log-based 10 polynucleotide copies per millimeter of plasma.

At step 410, a predicted viremia estimate from a sample is generated based on a combination of at least: the first parameter value, the second parameter value, and the third parameter value. In particular, the predicted viremia estimate value is generated by evaluating the following equation:

Predicted Viremia Estimate=10\hat{ }[I₀+(a₁)(frac.pos)+(a₂)(frac.cont)+(a₃)(log(est.vir,10))+(a₄)(frac.cont)(log(est.vir,10)).

The term “a₁” represents a respective estimated coefficient for the “frac.pos” parameter, which as explained above can range from about 0.527 to about 2.037. The term “a₂” represents a respective estimated coefficient for the “frac.cont” parameter, which as explained above can range from about −4.13 to about 12.652. The term “a₃” represents a respective estimated coefficient for the “log(est.vir, 10)” parameter, which as explained above can range from about 2.2 to about 6.73. The term “a₄” represents a respective estimated coefficient for the interaction between the “frac.cont” parameter and the “log(est.vir, 10)” parameter, which as explained above can range from about −14.804 to about −0.596. The term “I₀” represents an estimated initial intercept, which as explained above can range from about −6.24 to about −2.942.

Then at step 412, the predicted viremia estimate value is stored to a database by the computing system.

FIG. 5 of the drawings shows hardware 1300 associated with an exemplary computing system that may be used to implement methods (i.e. method 400) disclosed herein. The hardware 1300 typically includes at least one processor 1302 coupled to a memory 1304. The processor 1302 may represent one or more processors (e.g. microprocessors), and the memory 1304 may represent random access memory (RAM) devices comprising a main storage of the hardware 1300, as well as any supplemental levels of memory, e.g., cache memories, non-volatile or back-up memories (e.g. programmable or flash memories), read-only memories, etc. In addition, the memory 1304 may be considered to include memory storage physically located elsewhere in the hardware 1300, e.g. any cache memory in the processor 1302 as well as any storage capacity used as a virtual memory, e.g., as stored on a mass storage device 1310.

The hardware 1300 also typically receives a number of inputs and outputs for communicating information externally. For an interface with a user or operator, the hardware 1300 may include one or more user input devices 1306 (e.g., a keyboard, a mouse, imaging device, scanner, etc.) and one or more output devices 1308 (e.g., a Liquid Crystal Display (LCD) panel, a sound playback device (speaker)). To embody the present disclosure, the hardware 1300 may include at least one touch screen device (for example, a touch screen), an interactive whiteboard or any other device which allows the user to interact with a computer by touching areas on the screen.

For additional storage, the hardware 1300 may also include one or more mass storage devices 1310, e.g., a floppy or other removable disk drive, a hard disk drive, a Direct Access Storage Device (DASD), an optical drive (e.g. a Compact Disk (CD) drive, a Digital Versatile Disk (DVD) drive, etc.) and/or a tape drive, among others. Furthermore, the hardware 1300 may include an interface with one or more networks 1312 (e.g., a local area network (LAN), a wide area network (WAN), a wireless network, and/or the Internet among others) to permit the communication of information with other computers coupled to the networks. It should be appreciated that the hardware 1300 typically includes suitable analog and/or digital interfaces between the processor 1302 and each of the components 1304, 1306, 1308, and 1312 as is well known in the art.

The hardware 1300 operates under the control of an operating system 1314, and executes various computer software applications, components, programs, objects, modules, etc. to implement the techniques described above. In particular, the computer software applications will include the client dictionary application, in the case of the client user device. Moreover, various applications, components, programs, objects, etc., collectively indicated by reference 1316 in FIG. 5, may also execute on one or more processors in another computer coupled to the hardware 1300 via a network 1312 e.g., in a distributed computing environment, whereby the processing required to implement the functions of a computer program may be allocated to multiple computers over a network.

In general, the routines executed to implement the embodiments of the disclosure may be implemented as part of an operating system or a specific application, component, program, object, module or sequence of instructions referred to as “computer programs.” The computer programs typically comprise one or more instructions set at various times in various memory and storage devices in a computer, and that, when read and executed by one or more processors in a computer, cause the computer to perform operations necessary to execute elements involving the various aspects of the disclosure. Moreover, while the disclosure has been described in the context of fully functioning computers and computer systems, those skilled in the art will appreciate that the various embodiments of the disclosure are capable of being distributed as a program product in a variety of forms, and that the disclosure applies equally regardless of the particular type of computer-readable media used to actually effect the distribution. Examples of computer-readable media include but are not limited to recordable type media such as volatile and non-volatile memory devices, floppy and other removable disks, hard disk drives, optical disks (e.g., Compact Disk Read-Only Memory (CD-ROMs), Digital Versatile Disks (DVDs), flash memory, etc.), among others. Another type of distribution may be implemented as Internet downloads.

While certain exemplary embodiments have been described and shown in the accompanying drawings, it is to be understood that such embodiments are merely illustrative and not restrictive of the broad disclosure and that this disclosure is not limited to the specific constructions and arrangements shown and described, since various other modifications may occur to those ordinarily skilled in the art upon studying this disclosure. In an area of technology such as this, where growth is fast and further advancements are not easily foreseen, the disclosed embodiments may be readily modifiable in arrangement and detail as facilitated by enabling technological advancements without departing from the principals of the present disclosure.

In the foregoing specification, specific embodiments of the present disclosure have been described. However, one of ordinary skill in the art appreciates that various modifications and changes can be made without departing from the scope of the present disclosure as set forth in the claims below. Accordingly, the specification and figures are to be regarded in an illustrative rather than a restrictive sense, and all such modifications are intended to be included within the scope of present disclosure. The benefits, advantages, solutions to problems, and any element(s) that may cause any benefit, advantage, or solution to occur or become more pronounced are not to be construed as a critical, required, or essential features or elements of any or all the claims. The disclosure is defined solely by the appended claims including any amendments made during the pendency of this application and all equivalents of those claims as issued.

1. A method for predictably quantifying the amount of a polynucleotide in a volume of a sample via a computing system, the method comprising:
a) obtaining, by the computing system, a plurality of quantification results of the polynucleotide in the sample from an assay having a lower limit of detection;
b) generating, by the computing system, a first parameter value by determining the fraction of the results that have a positive result;
c) generating, by the computing system, a second parameter value by determining the fraction of results that have a positive and quantified result;
d) generating, by the computing system, a third parameter value by generating a nominal continuous viremia estimate for the sample and calculating the logarithm base 10 of the nominal continuous viremia estimate;
e) generating, by the computing system, a predicted number of the polynucleotide copies per the volume of the sample based on a combination of at least: the first parameter value, the second parameter value, and the third parameter value; and
f) storing the predicted number in a database.

2. The method of claim 1, wherein the assay is a quantitative polymerase chain reaction (PCR) assay.

3. The method of claim 1, wherein the plurality of quantification results correspond with detection and/or lack of detection of one or more fluorescent molecules.

4. The method of claim 3, wherein one of the one or more fluorescent molecules is 6-carboxyfluorescein.

5. The method of claim 3, wherein one of the one or more fluorescent molecules is tetrachlorofluorescein.

6. The method of claim 1, wherein the plurality of quantification results in step a) comprises ten or more quantification results.

7. The method of claim 1, wherein the nominal continuous viremia estimate of step d) is generated by at least:
a) assigning a value from 0 to 5 polynucleotide copies per milliliter of the sample to any negative result;
b) assigning a value from 5 to 10 polynucleotide copies per milliliter of the sample to any positive but unquantified result;
c) assigning a continuous value of polynucleotide copies per milliliter of the sample to each positive and quantified result; and
d) averaging all the assigned values.

8. The method of claim 1, wherein the nominal continuous viremia estimate of step d) is generated by at least:
a) assigning a value of 0 polynucleotide copies per milliliter of the sample to any negative result;
b) assigning a value of 5 polynucleotide copies per milliliter of the sample to any positive but unquantified result;
c) assigning a continuous value of polynucleotide copies per milliliter of the sample to each positive and quantified result; and
d) averaging all the assigned values.

9. The method of claim 1, wherein the assay is a Roche COBAS AmpliPrep/TAQMAN Assay.

10. The method of claim 9, wherein the positive result of step b) is a non-TND result.

11. The method of claim 9, wherein the nominal continuous viremia estimate of step d) is generated by at least:
a) assigning a value of 5 polynucleotide copies per milliliter of the sample to any negative (TND) result;
b) assigning a value of 10 polynucleotide copies per milliliter of the sample to any positive but unquantified result;
c) assigning a continuous value of polynucleotide copies per milliliter of the sample to any positive and quantified result; and
d) averaging all the assigned values.

12. The method of claim 1, wherein the predicted number of polynucleotides per milliliter of the sample is generated by evaluating the following formula: 10\hat{ }[I₀+(a₁)(frac.pos)+(a₂)(frac.cont)+(a₃)(log(est.vir,10))+(a₄)(frac.cont)(log(est.vir,10)).

13. The method of claim 12, wherein at is from about 0.527 to about 2.037, wherein a₂ is from about −4.13 to about 12.652, wherein a₃ is from about 2.2 to about 6.73, wherein a₄ is from about −14.804 to −0.596, and wherein I₀ is from about −6.24 to about −2.942.

14. The method of claim 12, wherein at is =1.282, wherein a₂ is =4.261, wherein a₃ is =4.465, wherein a₄ is =−7.770, and wherein I₀ is =−4.591.

15. The method of claim 1, wherein the polynucleotide comprises Human Immunodeficiency Virus (HIV) RNA.

16. The method of claim 1, wherein the sample comprises blood plasma.

17. The method of claim 1, wherein the sample is from a subject treated with suppressive antiretroviral therapy (ART).

18. The method of claim 1, wherein the predicted number of polynucleotide copies per milliliter of the sample is less than a lower detection limit of the assay.

19. The method of claim 1, wherein the predicted number of polynucleotide copies per milliliter of the sample is from about 0.2 to 5.8-fold of a quantified result from a mega-iSCA analysis of the sample.
